(19) United States
(12) Patent Application Publication
(10) Pub. No.: US 2024/0374809 A1
(43) Pub. Date: Nov. 14, 2024

(54) INTRAVENOUS POLE FOR IMPROVED SAFETY AND ACCURACY OF INTRAVENOUS MEDICATION ADMINISTRATION

(71) Applicant: University of Massachusetts, Boston, MA (US)

(72) Inventors: Karen Giuliano, Boston, MA (US); Juan Jiménez, Boston, MA (US); Jeannine Blake, Boston, MA (US)

(21) Appl. No.: 18/660,623

(22) Filed: May 10, 2024

Related U.S. Application Data
(60) Provisional application No. 63/465,705, filed on May 11, 2023.

(51) Int. Cl. A61M 5/14

(52) U.S. Cl. CPC A61M 5/1417; A61M 5/1415

(57) ABSTRACT

Various implementations include an intravenous (IV) therapy pole device. The device includes a pole, a first crossbar, and a second crossbar. The pole extends along a pole longitudinal axis. The first crossbar extends along a first longitudinal axis. The first crossbar is couplable to the pole such that the first longitudinal axis is transverse to the pole longitudinal axis. The second crossbar extends along a second longitudinal axis. The second crossbar is couplable to the pole such that the second longitudinal axis is transverse to the pole longitudinal axis. The first crossbar and the second crossbar are spaced apart along the pole longitudinal axis by a distance. The distance is in a range of 6 to 48 inches.

### CROSS-REFERENCE TO RELATED APPLICATIONS

This application claims priority to U.S. Provisional Application No. 63/465,705 filed May 11, 2023, which is herein incorporated by reference.

### BACKGROUND

Hospitalized patients are at high risk for adverse drug events associated with the delivery of life-critical intravenous (IV) medications. It is estimated that half a million hospitalized Americans will be harmed by preventable medication errors annually. Medication errors comprise 78% of all medical errors in intensive care, with 20% carrying a risk of death and 40% requiring intervention. IV smart pumps (IVSPs) are widely used to deliver IV medications and fluid during acute care hospitalization, and a significant source of medication error is caused by flow rate inaccuracy due to incorrect system setup when using linear peristaltic IVSPs. Linear peristaltic IVSPs represent approximately 90% of the IVSPs in current clinical use and their accurate flow is reliant on sufficient intake pressure, which is partly generated by the location of the IV bags above the IVSP. System setup that does not match manufacturer setup requirements can result in differences between the actual flow rate and programmed flow rate, even though the IVSP will continue to display the programmed flow rate, causing occult IV medication errors that are difficult to detect and contribute significantly to overall IV medication error.

### SUMMARY

Various implementations include an intravenous (IV) therapy pole device. The device includes a pole, a first crossbar, and a second crossbar. The pole extends along a pole longitudinal axis. The first crossbar extends along a first longitudinal axis. The first crossbar is couplable to the pole such that the first longitudinal axis is transverse to the pole longitudinal axis. The second crossbar extends along a second longitudinal axis. The second crossbar is couplable to the pole such that the second longitudinal axis is transverse to the pole longitudinal axis. The first crossbar and the second crossbar are spaced apart along the pole longitudinal axis by a distance. The distance is in a range of 6 to 48 inches.

In some implementations, the distance is in the range of 12 to 36 inches. In some implementations, the distance is 18 to 28 inches.

In some implementations, each of the first crossbar and the second crossbar is integrally and statically coupled to the pole.

In some implementations, the pole further comprises a first mounting portion configured for coupling the first crossbar to the pole and a second mounting portion configured for coupling the second crossbar to the pole.

In some implementations, each of the first crossbar and the second crossbar is slidably coupled to the pole. In some implementations, the pole includes at least one set of two or more distance markings. In some implementations, the two or more distance markings are spaced apart along the pole longitudinal axis. In some implementations, the pole includes one or more positive stops for indicating location of the first crossbar or the second crossbar along the pole.

In some implementations, the second crossbar includes one or more first bag hooks for coupling an IV bag to the second crossbar. In some implementations, the one or more first bag hooks includes four or more first bag hooks. In some implementations, the four or more first bag hooks includes six or more first bag hooks.

In some implementations, the second crossbar includes one or more bag markings. In some implementations, each of the one or more bag markings is different than any other bag marking. In some implementations, each of the one or more bag markings is disposed adjacent a different one of the one or more first bag hooks for identifying the different one of the one or more first bag hooks.

In some implementations, the second crossbar further includes one or more IV bag extensions having a bag extension longitudinal axis. In some implementations, each of the one or more IV bag extensions is coupled to an end of the second crossbar such that the second longitudinal axis and the bag extension longitudinal axis reside in a plane that is perpendicular to the pole longitudinal axis. In some implementations, the bag extension longitudinal axis is transverse to the second longitudinal axis. In some implementations, at least one of the one or more bag extensions are hingedly coupled to the end of the second crossbar.

In some implementations, the device further includes a third crossbar extending along a third longitudinal axis. In some implementations, the third crossbar is couplable to the pole such that the third longitudinal axis is transverse to the pole longitudinal axis. In some implementations, the third crossbar is disposed along the pole longitudinal axis above the first crossbar and above the second crossbar. In some implementations, the third crossbar and the second crossbar are spaced apart along the pole longitudinal axis by a secondary distance in a range of 4 to 20 inches. In some implementations, the secondary distance is in the range of 6 to 18 inches. In some implementations, the secondary distance is 8 to 16 inches.

In some implementations, the third crossbar includes one or more second bag hooks for coupling an IV bag to the third crossbar. In some implementations, the one or more second bag hooks includes four or more second bag hooks. In some implementations, the four or more second bag hooks includes six or more second bag hooks.

In some implementations, the third crossbar includes one or more secondary bag markings. In some implementations, each of the one or more secondary bag markings is different than any other secondary bag marking. In some implementations, each of the one or more secondary bag markings is disposed adjacent a different one of the one or more second bag hooks for identifying the different one of the one or more second bag hooks.

In some implementations, the second crossbar is a non-infusing IV bag crossbar. In some implementations, the third crossbar is an infusing IV bag crossbar. In some implementations, the non-infusing IV bag crossbar includes one or more first bag hooks for coupling a non-infusing IV bag to the non-infusing IV bag crossbar. In some implementations, the infusing IV bag crossbar includes one or more second bag hooks for coupling an infusing IV bag to the infusing IV bag crossbar.

In some implementations, the first crossbar includes one or more pump mounts for coupling an IV pump to the first crossbar. In some implementations, at least one of the one or more pump mounts is a swivel mount configured to allow an IV pump coupled to the at least one of the one or more pump mounts to swivel relative to the first crossbar.

In some implementations, the one or more pump mounts includes four or more pump mounts. In some implementations, the four or more pump mounts includes six or more pump mounts.

In some implementations, the first crossbar includes one or more pump markings. In some implementations, each of the one or more pump markings is different than any other pump marking. In some implementations, each of the one or more pump markings is disposed adjacent a different one of the one or more pump mounts for identifying the different one of the one or more pump mounts.

In some implementations, the first crossbar further includes one or more pump extensions having a pump extension longitudinal axis. In some implementations, each of the one or more pump extensions is coupled to an end of the first crossbar such that the first longitudinal axis and the first longitudinal axis reside in a plane that is perpendicular to the pole longitudinal axis. In some implementations, the pump extension longitudinal axis is transverse to the first longitudinal axis. In some implementations, at least one of the one or more pump extensions is hingedly coupled to the end of the first crossbar.

In some implementations, the first crossbar includes one or more pump mounts for coupling an IV pump to the first crossbar, and the second crossbar includes one or more first bag hooks for coupling an IV bag to the second crossbar. In some implementations, each of the one or more pump mounts is vertically aligned with a different one of the one or more first bag hooks relative to the pole longitudinal axis.

In some implementations, the device further includes a base coupled to an end of the pole such that the base is closer to the first crossbar than it is to the second crossbar. In some implementations, the base includes three or more casters.

In some implementations, the device further includes a vertical adjustment device for moving the first crossbar and the second crossbar along the pole longitudinal axis relative to the base. In some implementations, the vertical adjustment device includes a hydraulic pump. In some implementations, the base includes a foot pedal for causing the vertical adjustment device to move the first crossbar and the second crossbar along the pole longitudinal axis relative to the base. In some implementations, the pole includes a telescoping pole. In some implementations, the vertical adjustment device causes the telescoping pole to extend longitudinally.

In some implementations, the device further includes a handle.

In some implementations, the device further includes a power strip couplable to the device, the power strip including an electrical power input and two or more electrical power outputs that are each electrically couplable to an IV pump.

### BRIEF DESCRIPTION OF DRAWINGS

Example features and implementations are disclosed in the accompanying drawings. However, the present disclosure is not limited to the precise arrangements and instrumentalities shown.

FIG. 1 is a perspective view of an intravenous (IV) therapy pole device, according to one implementation.

FIG. 2 is a side view of the intravenous (IV) therapy pole device of FIG. 1.

FIG. 3 is a partial view of a portion of the intravenous (IV) therapy pole device of FIG. 1.

FIG. 4 is a partial view of a portion of the intravenous (IV) therapy pole device of FIG. 1.

### DETAILED DESCRIPTION

Some implementations of devices, systems, and methods disclosed herein provide for an innovative and novel multi-channel intravenous (IV) pole to facilitate correct usage of manufacturer setup requirements for: 1) the heights of the intravenous (IV) bags relative to the IV smart pumps (IVSPs); and 2) the IV smart pumps (IVSPs) locations relative to the patient, both of which are necessary for accurate fluid flow.

The devices, systems, and methods described herein provide for more repeatable IV smart pump placement. The devices, systems, and methods described herein can include an IV pole in which the height of the IV bags and IV smart pumps can be raised or lowered relative to the base. The IV pole can be adjusted such that the IV smart pump is placed at the level of the patient.

The devices, systems, and methods described herein also provide for more repeatable IV bag heights. The devices, systems, and method described herein provide for more repeatable IV bag heights for a single IV bag or a plurality of IV bags (e.g., a “non-infusing” IV bag and an “infusing” IV bag at a different height than the “non-infusing IV bag”). The IV bag should be hung above the pump, ensuring the top of the fluid level is a specified height (e.g., approximately between 22 and 26 inches for the Baxter IV smart pump; approximately 20 inches for the Alaris pump) above the center of the pump. For example, the infusing bag on the infusing crossbar may be spaced apart from the other crossbars such that the fluid level of the infusing IV bag is 18-26 inches above the middle of the IV pump. Depending on the type and shape of IV bags used, a pump to fluid level distance of 18-26 inches may correspond to a distance of 18-32 inches between the pump crossbar and the infusing crossbar. The devices, systems, and methods described herein can include mounts for the IV smart pumps (IVSP) and hangers for the IV bags that are set at the correct distance from the IVSP for a given IVSP to administer medication properly as specified by the manufacturer or other reliable source for medication administration. The IV bag hangers can be a set distance from the IVSP mounts or can be adjustable to certain distances that are proper for various IVSP models. In some implementations, the IVSP is coupled to a first crossbar below a second crossbar along an IV pole.

The devices, systems, and methods described herein also provide for more reliable infusion bag heights. In some implementations, a first IV bag is a “non-infusing IV bag” while a second IV bag is an “infusing IV bag” such that the “infusing” IV bag is disposed above the “non-infusing” IV bag. In some implementations, the first or “non-infusing” IV bag is located on a crossbar (e.g., a second crossbar) and is used for standard, continuous infusion of fluids or medications while the second or “infusing” IV bag is located on a separate crossbar (e.g., a third crossbar) and is being used to deliver medications, as it is used for periodic or intermittent IV infusion.

The infusing IV bag should also be a specified height (e.g., 24 inches for the Baxter IVSP; 20 inches for the Alaris pump) above the center of the pump. The infusing IV bag may be placed above the non-infusing IV bag (or the non-infusing bag lowered below the infusing IV bag) to create a pressure difference that ensures infusion (e.g., a minimum of 9.5 inches between the non-infusing and infusing IV bags). If the pressure difference is not sufficient, there will be concurrent flow from both the non-infusing and infusing IV bags, or no flow at all from the infusing IV bag, both of which result in an undetected medication error. The devices, systems, and methods described herein can include hangers for the infusing IV bags that are set at the correct distance from the IV smart pumps (IVSPs) for a given IVSP to administer medication properly and at the correct distance from the non-infusing IV bags. The infusing IV bag hangers can be a set distance from the IVSP mounts and non-infusing IV bags or can be adjustable to certain distances that are proper for various IVSP models.

In some implementations, the non-infusing IV bag is moved from a lower IV bag hanger to a higher IV bag hanger (e.g., when the infusion of the “infusing IV bag” is complete). In such an implementation, the non-infusing IV bag being moved from a lower to a higher IV bag hanger moves from a “non-infusing” to an “infusing” location (e.g., from a “non-infusing” IV bag hanger or crossbar to an “infusing” IV bag hanger or crossbar). In some implementations, the non-infusing IV bag may be moved back to a lower IV bag hanger to accommodate a new infusing IV bag on the higher, “infusing” location, hanger, or crossbar.

Disclosed herein are novel IV poles which facilitate the correct usage of IV smart pump system set up in the clinical setting. The devices disclosed herein transform the current manual process, which relies on IV poles that provide no setup guidance to the clinician to mitigate the risk of IV smart pump (IVSP) setup errors.

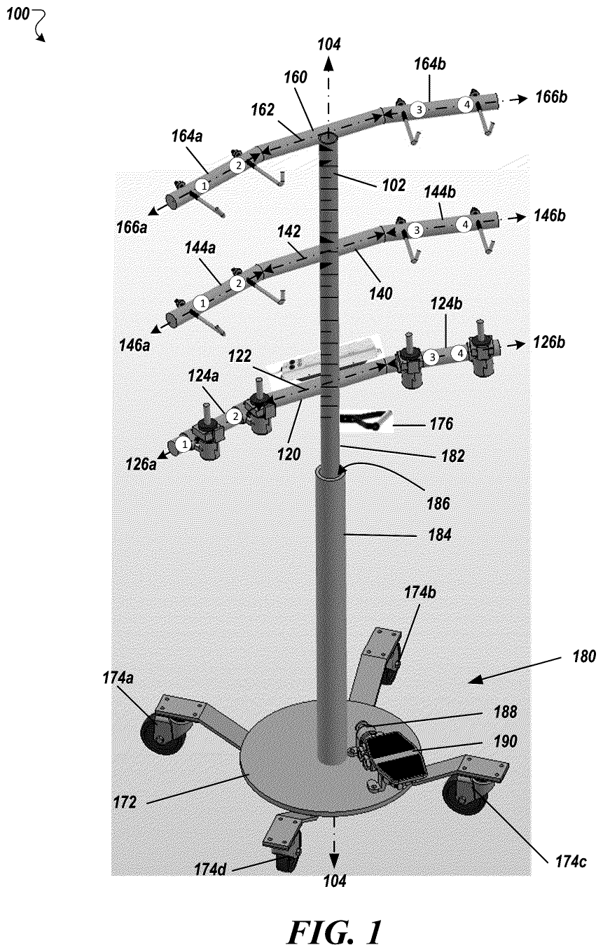

FIGS. 1-4 show an intravenous (IV) therapy pole device 100. The device 100 includes a pole 102, a first crossbar 120, a second crossbar 140, a third crossbar 160, and a base 172. In some implementations, the second crossbar is a “non-infusing” crossbar and the third crossbar is an “infusing” crossbar.

The pole 102 extends along a pole longitudinal axis 104. In some implementations, the device includes more than one pole (e.g., a device having two poles separated by a distance in a direction perpendicular to the pole longitudinal axis). In some implementations, the device includes two upper poles separated by a distance on one end and connected to each other on the other end to form a single pole on the lower end of the poles, the single pole being coupled to a base.

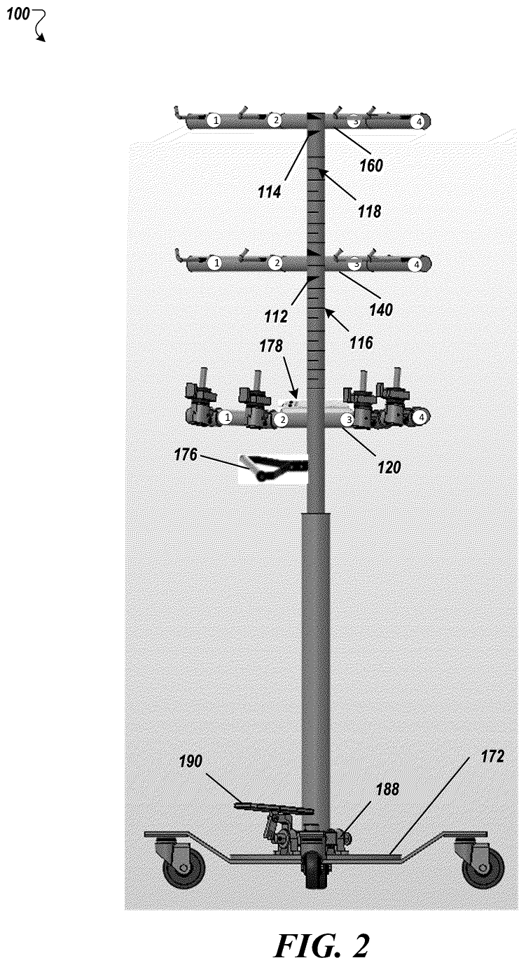

The pole 102 includes two sets of positive stops 112, 114, as shown in FIG. 2. Each of the positive stops 112, 114 in the two sets places the second crossbar 140 or the third crossbar 160 in a position such that the IV bags coupled to the crossbars are disposed at the preferred distance for a given model IV pump 130. Thus, the pole 102 can have positive stops 112, 114 for one or more IV pump models. In addition, the pole 102 can include positive stops 112, 114 at evenly spaced intervals, such as every inch or every five centimeters. In other implementations, the pole includes positive stops (e.g., mechanical or electromechanical) for other crossbars or more than one crossbar of the device.

The pole 102 further includes a first set of distance markings 116 and a second set of distance markings 118. The first set of distance markings 116 and the second set of distance markings 118 are spaced apart along the pole longitudinal axis 104. The first set of distance markings 116 indicate the model of an IV pump 130 associated with a certain position on the pole 102 relative to the first crossbar 120 or they can indicate a measurement of the distance between the position and the first crossbar 120. In some implementations, the first set of distance markings is a “non-infusing set of distance markings” and the second set of distance markings is an “infusing set of distance markings.” In some implementations, the distance markings are based on a desired distance between an IV bag and an IV pump (e.g., a manufacturer's recommended height of the fluid in the IV bag).

Although the pole 102 shown in FIGS. 1-4 includes two sets of positive stops 112, 114, in some implementations, the pole includes one or more sets of positive stops. In some implementations, the pole does not include positive stops. Although the pole shown in FIGS. 1-4 includes a first set of distance markings 116 and a second set of distance markings 118, in some implementations, the pole includes any number of one or more sets of two or more distance markings spaced apart along the pole longitudinal axis.

The device 100 further includes a handle 176 for moving the device 100, the handle being coupled to the pole 102. In some implementations, the handle is coupled to second telescoping segment 184. The device 100 also includes an electrical power strip 178 attachable to the first crossbar 120. The electrical power strip 178 includes one input and four electrical power outputs that are each electrically couplable to an IV pump 130. However, in some implementations, the pole can include any number of power inputs that are in electrical communication with any number of electrical outputs. In some implementations, the electrical power strip is a non-integrated power strip (e.g., an off-the-shelf power strip) attachable/removable from a portion of the device (e.g., the first crossbar).

The first crossbar 120 extends along a first longitudinal axis 122. The first crossbar 120 is couplable to the pole 102 such that the first longitudinal axis 122 is transverse to the pole longitudinal axis 104. The first crossbar 120 shown in FIGS. 1-4 is slidably coupled to the pole 102 to allow the first crossbar 120 to be adjusted relative to the pole longitudinal axis 104.

The first crossbar 120 includes two pump extensions 124a, 124b each having a pump extension longitudinal axis 126a, 126b. Each of the two pump extensions 124a, 124b is coupled to a separate end of the first crossbar 120 such that the first longitudinal axis 122 and the pump extension longitudinal axis 126a, 126b of each pump extension 124a, 124b reside in a plane that is perpendicular to the pole longitudinal axis 104. Each of the pump extension longitudinal axes 126a, 126b is transverse to the first longitudinal axis 122 to form bends near the ends of the first crossbar 120. Although the pump extensions 124a, 124b are statically coupled to the ends of the first crossbar 120, in some implementations, at least one of the pump extensions is hingedly coupled to the end of the first crossbar to allow the first crossbar to be folded into a smaller shape. In other implementations, the pump extensions and the first crossbar both extend along the first longitudinal axis.

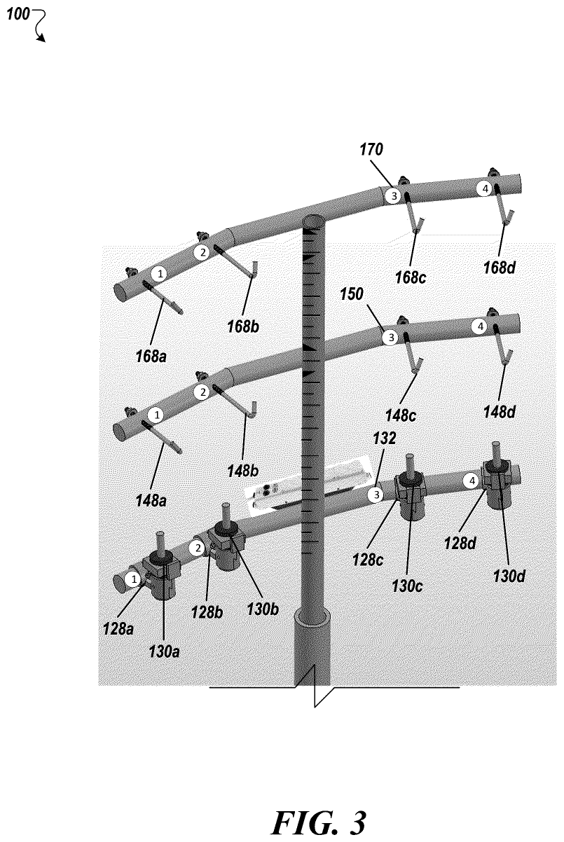

As shown in FIG. 3, the first crossbar 120 includes four pump mounts 128a-d for coupling an IV pump 130 to the first crossbar 120. When the first crossbar 120 is coupled to the pole 102, two of the pump mounts 128a, 128b are disposed on one side of the pole 102 and the other two pump mounts 128c, 128d are disposed on the other side of the pole 102. Although the first crossbar 120 shown in FIGS. 1-4 includes four pump mounts 128a-d, in some implementations, the first crossbar includes six pump mounts with three pump mounts disposed on one side of the pole and the other three pump mounts disposed on the other side of the pole. In some implementations, the first crossbar includes any number of one or more pump mounts for coupling an IV pump to the first crossbar (e.g., 2 pumps total). In some implementations, the first crossbar is curved. Throughout this disclosure, “pump crossbar” or “first crossbar” may refer to the assembly comprising the first crossbar 120 and the two pump extensions 124a, 124b coupled thereto.

Each of the four pump mounts 128a-d shown in FIGS. 1-4 is a swivel mount configured to allow an IV pump 130 coupled to each of the pump mounts 128a-d to swivel relative to the first crossbar 120 (e.g., the pump mounts can swivel 45 degrees in every direction relative to the first crossbar). However, in some implementations, any number of one or more of the pump mounts is a swivel mount. In some implementations, none of the pump mounts are swivel mounts.

In some implementations, one of the crossbars and/or its associated extensions (e.g., the first crossbar and the two pump extensions) further include a holder on the distal end (e.g., the distal end of each pump extension). In some implementations, the holder on the distal end of an extension is a holder for a hemodynamic transducer or other device. In some implementations, both ends of the first crossbar (e.g., the distal ends of each of the two pump extensions from the first crossbar) include a holder for a hemodynamic transducer. In some implementations, the holder for the hemodynamic transducer is a retractable hook. In some implementations, a first holder for the hemodynamic transducer is disposed one of the pump crossbar or the non-infusing crossbar, and a second holder is disposed on the other of the non-infusing crossbar or the pump crossbar, the second holder configured to hold a pressure bag. In some implementations, the device further includes at least one hemodynamic transducer (e.g., for measuring and monitoring one or more of blood pressure, blood flow, heart rate, or related cardiac variables).

The second crossbar 140 (e.g., a “non-infusing” IV bag crossbar) extends along a second longitudinal axis 142. The second crossbar 140 is couplable to the pole 102 such that the second longitudinal axis 142 is transverse to the pole longitudinal axis 104. The second crossbar 140 is slidably coupled to pole 102 and is releasably engageable with one of the sets of positive stops 112. The first crossbar 120 and the second crossbar 140 shown in FIGS. 1-4 are spaced apart along the pole longitudinal axis 104 by a distance of 18 to 28 inches. However, in some implementations, the first crossbar and the second crossbar are spaced apart along the pole longitudinal axis by a distance in a range of 12 to 36 inches. In some implementations, the first crossbar and the second crossbar are spaced apart along the pole longitudinal axis by a distance in a range of 6 to 48 inches.

The second crossbar 140 includes two first IV bag extensions 144a, 144b each having a first IV bag extension longitudinal axis 146a, 146b. Each of the two first IV bag extensions 144a, 144b is coupled to a separate end of the second crossbar 140 such that the first IV bag longitudinal axis 142 and the first IV bag extension longitudinal axis 146a, 146b of each first IV bag extensions 144a, 144b reside in a plane that is perpendicular to the pole longitudinal axis 104. Each of the first IV bag extension longitudinal axes 146a, 146b is transverse to the first IV bag longitudinal axis 142 to form bends near the ends of the second crossbar 140. Although the first IV bag extensions 144a, 144b are statically coupled to the ends of the second crossbar 140, in some implementations, at least one of the first IV bag extensions is hingedly coupled to the end of the second crossbar to allow the second crossbar to be folded into a smaller shape. In some implementations, the second crossbar is curved. In some implementations, the second crossbar and the two first IV bag extensions both extend along the second longitudinal axis. Throughout this disclosure, “non-infusing crossbar” or “second crossbar” may refer to the assembly comprising the second crossbar 140 and the two first IV bag extensions 144a, 144b coupled thereto.

The second crossbar 140 includes four first bag hooks 148a-d for coupling an IV bag (not shown) (e.g., a “non-infusing IV bag”) to the second crossbar 140. Specifically, the four first bag hooks 148a-d are coupled to the two first IV bag extensions 144a, 144b. However, in other implementations, the bag hooks may be coupled directly to the crossbar (e.g., a central portion of the crossbar). When the second crossbar 140 is coupled to the pole 102, two of the first bag hooks 148a, 148b are disposed on one side of the pole 102 and the other two first bag hooks 148c, 148d are disposed on the other side of the pole 102. Although the second crossbar 140 shown in FIGS. 1-4 includes four first bag hooks 148a-d, in some implementations, the second crossbar includes six first bag hooks with three first bag hooks disposed on one side of the pole and the other three first bag hooks disposed on the other side of the pole. In some implementations, the second crossbar includes any number of one or more first bag hooks for coupling an IV bag to the second crossbar (e.g., 2 bag hooks). In some implementations, the second crossbar is curved. In some implementations, the second crossbar is coupled to one side of the pole while the first crossbar is coupled to an opposite side of the pole. In some implementations, the opposing sides of coupling for the first and second crossbar prevent an IV bag hanging from the non-infusing crossbar from interfering with a pump on the pump crossbar.

The third crossbar 160 (e.g., an “infusing IV bag crossbar”) extends along a third longitudinal axis 162. The third crossbar 160 is couplable to the pole 102 such that the third longitudinal axis 162 is transverse to the pole longitudinal axis 104. The third crossbar 160 and the second crossbar 140 shown in FIGS. 1-4 are spaced apart along the pole longitudinal axis 104 by a secondary distance of 8 to 16 inches. However, in some implementations, the third crossbar and the second crossbar are spaced apart along the pole longitudinal axis by a secondary distance in a range of 6 to 18 inches. In some implementations, the third crossbar and the second crossbar are spaced apart along the pole longitudinal axis by a secondary distance in a range of 4 to 20 inches.

The third crossbar 160 includes two second IV bag extensions 164a, 164b each having a second IV bag extension longitudinal axis 166a, 166b. Each of the two second IV bag extensions 164a, 164b is coupled to a separate end of the third crossbar 160 such that the third longitudinal axis 162 and the second IV bag extension longitudinal axis 166a, 166b of each second IV bag extensions 164a, 164b reside in a plane that is perpendicular to the pole longitudinal axis 104. Each of the second IV bag extension longitudinal axes 166a, 166b is transverse to the third longitudinal axis 162 to form bends near the ends of the third crossbar 160. Although the second IV bag extensions 164a, 164b are statically coupled to the ends of the third crossbar 160, in some implementations, at least one of the second IV bag extensions is hingedly coupled to the end of the third crossbar to allow the third crossbar to be folded into a smaller shape. In some implementations, the third crossbar is curved. In some implementations, the third crossbar and the two second IV bag extensions both extend along the third longitudinal axis.

The third crossbar 160 includes four second bag hooks 168a-d for coupling an IV bag (not shown) (e.g., an “infusing IV bag”) to the third crossbar 160. When the third crossbar 160 is coupled to the pole 102, two of the second bag hooks 168a, 168b are disposed on one side of the pole 102 and the other two second bag hooks 168c, 168d are disposed on the other side of the pole 102. Although the third crossbar 160 shown in FIGS. 1-4 includes four second bag hooks 168a-d, in some implementations, the third crossbar includes six second bag hooks with three second bag hooks disposed on one side of the pole and the other three second bag hooks disposed on the other side of the pole. In some implementations, the third crossbar includes any number of one or more second bag hooks for coupling an IV bag to the third crossbar (e.g., 2 bag hooks). In some implementations, the third crossbar is curved. Throughout this disclosure, “infusing crossbar” or “third crossbar” may refer to the assembly comprising the third crossbar 160 and the two second IV bag extensions 164a, 164b coupled thereto.

In some implementations, the device does not include a third crossbar or an “infusing IV bag crossbar”. In some implementations, the device includes any number of IV bag crossbars and any number of first crossbars.

A first length of the pump crossbar is defined from a distal end of the first pump extension 124a and a distal end of the second pump extension 124b on either side of the first crossbar 120. A second length of the non-infusing crossbar is defined from a distal end of the first IV bag extension 144a to a distal end of the first IV bag extension 144b on either side of the second crossbar 140. A third length of the infusing crossbar is defined from a distal end of the second IV bag extension 164a to a distal end of the second IV bag extension 164b on either side of the third crossbar 160. Each of the first length, the second length, and the third length may be between 20 and 36 inches (e.g., between 26 and 30 inches). In some implementations, the first, second, and third lengths are identical to ensure proper alignment of IV bags and pumps.

For the device shown in FIGS. 1-4, each of the four pump mounts 128a-d is vertically aligned with a different one of the four first bag hooks 148a-d and a different one of the four second bag hooks 168a-d relative to the pole longitudinal axis 104. The first crossbar 120 includes a first marking 132 disposed adjacent a different one of the pump mounts 128a-d. The second crossbar 140 includes a second marking 150 disposed adjacent a different one of the first bag hooks 148a-d. The third crossbar 160 includes a third marking 170 disposed adjacent a different one of the second bag hooks 168a-d. Each of the one or more second markings 150 is different than any other second markings 150. The one or more third markings 170 and the first markings 132 each match the second marking 150 located vertically aligned with it. In use, the second markings 150, the third markings 170, and the first markings 132 allow a user to easily identify which IV bag(s) should be fluidly coupled to a specific IV pump. For example, in FIG. 3, each of the bag hook 148a, the second bag hook 168a, and the first pump mount 128a includes a corresponding numerical marking of “1”. In other implementations, the markings have a different numeral, letter, shape, color, or combination of the above to visually and/or texturally mark the different groups IV bags and corresponding pumps.

Although the first crossbar 120 of FIGS. 1-4 is slidably coupled to the pole 102, in some implementations, the pole includes a first mounting portion configured for coupling the first crossbar to the pole. Although the second crossbar 140 is slidably coupled to the pole 102, in some implementations, the pole further includes a second mounting portion configured for coupling the second crossbar to the pole. Although the third crossbar 160 is slidably coupled to the pole 102, in some implementations, the pole further includes a third mounting portion configured for coupling the third crossbar to the pole. In some implementations, the mounting portion is a mechanical coupling (e.g., a bracket) configured to accept the corresponding crossbar.

The base 172 is coupled to the lower end of the pole 102 such that the base 172 is closer to the first crossbar 120 than it is to the second crossbar 140. The base 172 includes four casters 174a-d, but in some implementations, the base includes any number of three or more casters. In use, a user may utilize the handle 176 to easily move/roll the device 100 on the casters 174a-d. In some implementations, the base is heavy and/or wide enough for adequate stability of the IV therapy pole device (e.g., such that a device loaded with IV bags and other equipment will not fall over).

The device 100 also includes a vertical adjustment device 180 for moving each of the first crossbar 120, the second crossbar 140, and the third crossbar 160 along the pole longitudinal axis 104 relative to the base 172. For example, this allows a user to adjust the IV pumps 130 that are coupled to the first crossbar 120 to the height of the heart of the patient without changing the relative distances between the first crossbar 120, the second crossbar 140, and the third crossbar 160. For example, the pole 102 is adjustable so that the first crossbar 120 is movable between 28-48 inches above the base 172 (e.g., adjustable based on the height of the patient's heart).

The pole 102 shown in FIGS. 1-4 is a telescoping pole having a first telescoping segment 182 and a second telescoping segment 184. The second telescoping segment 184 defines an axially extending opening 186, and the first telescoping segment 182 is sized to be slidingly disposed within the opening 186. The first telescoping segment 182 includes the locations for the first crossbar 120, the second crossbar 140, and the third crossbar 160. In some implementations, the first telescoping segment defines the axially extending opening and the second telescoping segment is sized to be slidingly disposed within the opening.

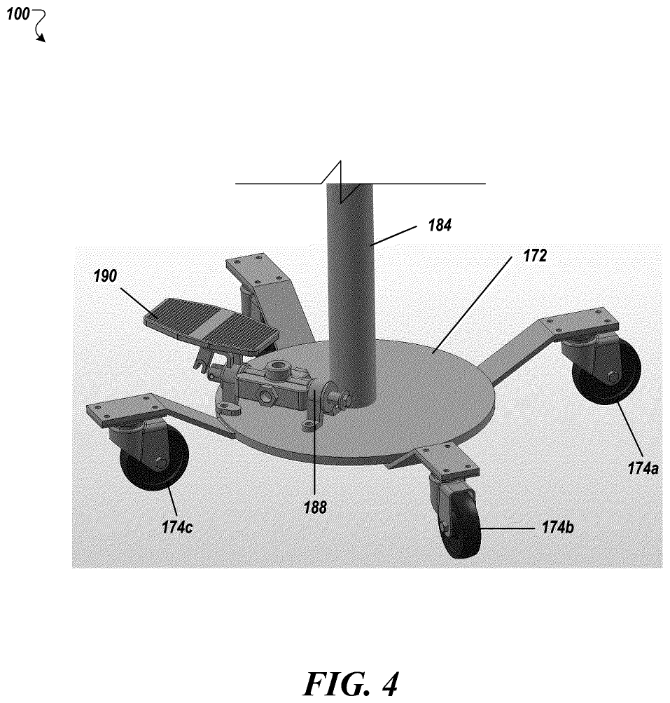

The vertical adjustment device 180 includes a hydraulic pump 188 that causes the first telescoping segment 182 to extend longitudinally from the opening 186 of the second telescoping segment 184. As seen in more detail in FIG. 4, the base 172 includes a foot pedal 190 for causing the hydraulic pump 188 of the vertical adjustment device 180 to extend the first telescoping segment 182 longitudinally to move the first crossbar 120, the second crossbar 140, and the third crossbar 160 along pole longitudinal axis 104 relative to the base 172. In some implementations, the foot pedal includes any standard pedal or lift mechanism (e.g., a continuous metal bar with a rubber section for grip, such as that commonly found on a hair salon chair or other chair device with a hydraulic lift mechanism). In some implementations, the hydraulic pump is disposed within a portion of the second telescoping segment (e.g., axially aligned with the pole).

In some implementations, a position indicator is provided on the first telescoping segment, viewable via an opening in the second telescoping segment. For example, an indicator (e.g., a green/yellow/red symbol) may be visible through the opening in the second telescoping segment based on the desired height of the pole. In some implementations, the set or desired height of the first telescoping segment of the pole corresponds to a green position indicator aligned with the opening in the second telescoping segment, while a misalignment or incorrect height corresponds to a yellow or red position indicator. The position indicator and/or the corresponding opening may be adjustable or selectable based on a desired height.

Further disclosed herein are methods for improving safety of intravenous (IV) medication administration. Such methods include providing an intravenous (IV) therapy pole device having a plurality of crossbars. In some implementations, the method further includes placing or attaching a pump to one of the plurality of crossbars and placing or attaching an IV bag to another one of the plurality of crossbars (e.g., to a bag hook on one of the plurality of crossbars). In some implementations, the method includes placing or attaching an IV bag to a crossbar at a first distance above the pump, the first distance being in a range of 6 to 48 inches. In some implementations, the method includes placing or attaching a second IV bag to a crossbar at a distance above the pump and a second distance above the first IV bag, the second distance being in a range of 4 to 20 inches. In some implementations, the method further includes vertically adjusting the IV therapy pole to a first height along a longitudinal axis of the IV therapy pole.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. An intravenous (IV) therapy pole device, the device comprising:
a pole extending along a pole longitudinal axis;
a first crossbar extending along a first longitudinal axis, wherein the first crossbar is couplable to the pole such that the first longitudinal axis is transverse to the pole longitudinal axis; and
a second crossbar extending along a second longitudinal axis, wherein the second crossbar is couplable to the pole such that the second longitudinal axis is transverse to the pole longitudinal axis,
wherein the first crossbar and the second crossbar are spaced apart along the pole longitudinal axis by a distance, wherein the distance is in a range of 6 to 48 inches.

2-3. (canceled)

4. The device of claim 1, wherein each of the first crossbar and the second crossbar are slidably coupled to the pole.

5. The device of claim 4, wherein the pole includes at least one set of two or more distance markings, wherein the two or more distance markings are spaced apart along the pole longitudinal axis.

6. (canceled)

7. The device of claim 1, wherein the second crossbar comprises one or more first bag hooks for coupling an IV bag to the second crossbar.

8-9. (canceled)

10. The device of claim 7, wherein the second crossbar comprises one or more bag markings, wherein each of the one or more bag markings is different than any other bag marking, wherein each of the one or more bag markings is disposed adjacent a different one of the one or more first bag hooks for identifying the different one of the one or more first bag hooks.

11. The device of claim 1, wherein the second crossbar further includes one or more IV bag extensions having a bag extension longitudinal axis, wherein each of the one or more IV bag extensions is coupled to an end of the second crossbar such that the second longitudinal axis and the bag extension longitudinal axis reside in a plane that is perpendicular to the pole longitudinal axis, wherein the bag extension longitudinal axis is transverse to the second longitudinal axis.

12. The device of claim 11, wherein at least one of the one or more IV bag extensions is hingedly coupled to the end of the second crossbar.

13. The device of claim 1, further comprising a third crossbar extending along a third longitudinal axis, wherein the third crossbar is couplable to the pole such that the third longitudinal axis is transverse to the pole longitudinal axis.

14. The device of claim 13, wherein the third crossbar is disposed along the pole longitudinal axis above the first crossbar and above the second crossbar.

15. The device of claim 14, wherein the third crossbar and the second crossbar are spaced apart along the pole longitudinal axis by a secondary distance, wherein the secondary distance is in a range of 4 to 20 inches.

16. The device of claim 13, wherein the third crossbar comprises one or more second bag hooks for coupling an IV bag to the third crossbar.

17-18. (canceled)

19. The device of claim 16, wherein the third crossbar comprises one or more secondary bag markings, wherein each of the one or more secondary bag markings is different than any other secondary bag marking, wherein each of the one or more secondary bag markings is disposed adjacent a different one of the one or more second bag hooks for identifying the different one of the one or more second bag hooks.

20. The device of claim 13, wherein the second crossbar is a non-infusing IV bag crossbar and the third crossbar is an infusing IV bag crossbar,
wherein the non-infusing IV bag crossbar comprises one or more first bag hooks for coupling a non-infusing IV bag to the non-infusing IV bag crossbar, and
wherein the infusing IV bag crossbar comprises one or more second bag hooks for coupling an infusing IV bag to the infusing IV bag crossbar.

21. The device of claim 1, wherein the first crossbar includes one or more pump mounts for coupling an IV pump to the first crossbar.

22-24. (canceled)

25. The device of claim 21, wherein the first crossbar comprises one or more pump markings, wherein each of the one or more pump markings is different than any other pump marking, wherein each of the one or more pump markings is disposed adjacent a different one of the one or more pump mounts for identifying the different one of the one or more pump mounts.

26. The device of claim 1, wherein the first crossbar further includes one or more pump extensions having a pump extension longitudinal axis, wherein each of the one or more pump extensions is coupled to an end of the first crossbar such that the first longitudinal axis and the pump extension longitudinal axis reside in a plane that is perpendicular to the pole longitudinal axis, wherein the pump extension longitudinal axis is transverse to the first longitudinal axis.

27. (canceled)

28. The device of claim 1, wherein the first crossbar includes one or more pump mounts for coupling an IV pump to the first crossbar, and the second crossbar includes one or more first bag hooks for coupling an IV bag to the second crossbar, wherein each of the one or more pump mounts is vertically aligned with a different one of the one or more first bag hooks relative to the pole longitudinal axis.

29. The device of claim 1, further comprising a base coupled to an end of the pole such that the base is closer to the first crossbar than it is to the second crossbar, wherein the base includes three or more casters.

30. (canceled)

31. The device of claim 29, further comprising a vertical adjustment device disposed on the base of the device, the vertical adjustment device comprising a hydraulic pump and a foot pedal for moving the first crossbar and the second crossbar along the pole longitudinal axis relative to the base.

32-33. (canceled)

34. The device of claim 31, wherein the pole comprises a telescoping pole, wherein the vertical adjustment device causes the telescoping pole to extend longitudinally.

35-36. (canceled)
